(19) United States
(12) Patent Application Publication
(10) Pub. No.: US 2006/0025786 A1
(43) Pub. Date: Feb. 2, 2006

(54) Method for autologous transplantation

(75) Inventors: Bruno M. Giannetti, Bonn (DE); Samuel S. Asculai, Ontario (CA); Ahmed Idouraine, Chandler, AZ (US)

Correspondence Address: MORGAN, LEWIS & BOCKIUS LLP, 1701 MARKET STREET, PHILADELPHIA, PA, 19103-2921, US

(73) Assignee: Verigen Transplantation Service International (VTSI) AG

(21) Appl. No.: 10/962,868

(22) Filed: Oct. 12, 2004

Related U.S. Application Data
(63) Continuation of application No. 10/121,249, filed on Apr. 12, 2002, now abandoned.
Application No. 10/121,249 is a continuation-in-part of application No. 10/093,129, filed on Mar. 6, 2002, now Pat. No. 6,599,301.
Application No. 10/093,129 is a continuation of application No. 10/090,922, filed on Mar. 5, 2002, now Pat. No. 6,592,599.
Application No. 10/090,922 is a continuation of application No. 10/091,006, filed on Mar. 4, 2002, now Pat. No. 6,599,300.
Application No. 10/091,006 is a continuation of application No. 10/055,105, filed on Jan. 23, 2002, now Pat. No. 6,592,598.
Application No. 10/055,105 is a continuation of application No. 09/690,252, filed on Oct. 17, 2000, now Pat. No. 6,379,367.
Application No. 09/690,252 is a continuation of application No. 09/320,246, filed on May 26, 1999, now Pat. No. 6,283,980.
Application No. 09/320,246 is a continuation of application No. 09/088,142, filed on Jun. 1, 1998, now Pat. No. 6,120,514.
Application No. 09/088,142 is a continuation of application No. 08/857,090, filed on May 15, 1997, now Pat. No. 5,989,269.
Application No. 08/857,090 is a continuation-in-part of application No. 08/704,891, filed on Aug. 30, 1996, now Pat. No. 5,759,190.

(51) Int. Cl. A61B 17/08

(52) U.S. Cl. 606/151

(57) ABSTRACT

The present invention describes various support matrices to which cells can adhere and proliferate. Such support matrices are useful for implantation in a wound site to promote healing and regeneration of damaged tissue. The present invention further describes an article including a membrane having at least one layer having a porous surface and also including submucosal intestine tissue, and cells adhered to the layer. The present invention further describes that the cells adhered to the layer include chondrocyte cells.

### CROSS-REFERENCE TO RELATED APPLICATIONS

This application is a continuation-in-part of U.S. patent application Ser. No. 10/093,129, filed Mar. 6, 2002, which is a continuation of U.S. patent application Ser. No. 10/090,922, filed Mar. 5, 2002, which is a continuation of U.S. patent application Ser. No. 10/091,066, filed Mar. 4, 2002, which is a continuation of U.S. patent application Ser. No. 10/055,105, filed Jan. 23, 2002, which is a continuation of U.S. patent application Ser. No. 09/690,252, filed Oct. 17, 2000, which is a continuation of U.S. patent application Ser. No. 09/320,246, filed May 26, 1999 (now U.S. Pat. No. 6,283,980), which is a continuation of U.S. patent application Ser. No. 09/088,142, filed Jun. 1, 1998 (now U.S. Pat. No. 6,120,514), which is a continuation of U.S. patent application Ser. No. 08/857,090, filed May 15, 1997 (now U.S. Pat. No. 5,989,269), which is a continuation-in-part of U.S. patent application Ser. No. 08/704,891, filed Aug. 30, 1996 (now U.S. Pat. No. 5,759,190), and this application claims priority to provisional U.S. Application No. 60/283,253, filed Apr. 12, 2001, all of which are hereby incorporated by reference in their entirety.

### FIELD OF THE INVENTION

The present invention relates to the field of chondrocyte cell transplantation, bone and cartilage grafting, healing, joint repair and the prevention of arthritic pathologies. In particular, the present invention is directed to new methods and instruments for chondrocyte cell transplantation and cartilage regeneration.

### BACKGROUND OF THE INVENTION

More than 500,000 arthroplastic procedures and total joint replacements are performed each year in the United States. Approximately the same number of similar procedures are performed in Europe. Included in these numbers are about 90,000 total knee replacements and around 50,000 procedures to repair defects in the knee per year (In: Praemer A., Furner S., Rice, D. P., Musculoskeletal conditions in the United States, Park Ridge, Ill.: American Academy of Orthopaedic Surgeons, 1992, 125). A method for regeneration-treatment of cartilage would be most useful, and could be performed at an earlier stage of a joint damage, thus reducing the number of patients needing artificial joint replacement surgery. With such preventative methods of treatment, the number of patients developing osteoarthritis would also decrease.

Techniques used for resurfacing the cartilage structure in joints have mainly attempted to induce the repair of cartilage using subchondral drilling, abrasion and other methods whereby there is excision of diseased cartilage and subchondral bone, leaving vascularized cancellous bone exposed (Insall, J., Clin. Orthop. 1974, 101:61; Ficat R. P. et al, Clin Orthop. 1979, 144:74; Johnson L. L., In: (McGinty J. B., Ed.) Operative Arthroscopy, New York: Raven Press, 1991, 341).

Coon and Cahn (1966, Science 153: 1116) described a technique for the cultivation of cartilage synthesizing cells from chick embryo somites. Later Cahn and Lasher (1967, PNAS USA 58: 1131) used the system for analysis of the involvement of DNA synthesis as a prerequisite for cartilage differentiation. Chondrocytes respond to both EGF and FGF by growth (Gospodarowicz and Mescher, 1977, J. Cell Physiology 93: 117), but ultimately lose their differentiated function (Benya et al., 1978, Cell 15: 1313). Methods for growing chondrocytes were described and are principally being used with minor adjustments as described by Brittberg, M. et al. (Brittberg, M. et al., New Engl. J. Med. 1994, 331:889). Cells grown using these methods were used as autologous transplants into knee joints in patients. Additionally, Kolettas et al. examined the expression of cartilage-specific molecules such as collagens and proteoglycans under prolonged cell culturing. They found that despite morphological changes during culturing in monolayer cultures (Aulthouse, A. et al., In Vitro Cell Dev. Biol., 1989, 25:659; Archer, C. et al., J. Cell Sci. 1990, 97:361; Haanselmann, H. et al., J. Cell Sci. 1994, 107:17; Bonaventure, J. et al., Exp. Cell Res. 1994, 212:97) when compared to suspension cultures grown over agarose gels, alginate beads or as spinner cultures (retaining a round cell morphology) the expressed markers such as types II and IX collagens and the large aggregating proteoglycans, aggrecan, versican and link protein did not change. (Kolettas, E. et al, J. Cell Science 1995, 108:1991).

The articular chondrocytes are specialized mesenchymal derived cells found exclusively in cartilage. Cartilage is an avascular tissue whose physical properties depend on the extracellular matrix produced by the chondrocytes. During endochondral ossification chondrocytes undergo a maturation leading to cellular hypertrophy, characterized by the onset of expression of type X collagen (Upholt, W. B. and Olsen, R. R., In: Cartilage Molecular Aspects (Hall, B & Newman, 5, Eds.) CRC Boca Raton 1991, 43; Reichenberger, E. et al., Dev. Biol. 1991, 148:562; Kirsch, T. et al., Differentiation, 1992, 52:89; Stephens, M. et al., J. Cell Sci. 1993, 103:1111).

Despite the advances in cultivating chondrocytes, and manipulating bone and cartilage, there has not been great success with the attempts to transplant cartilage or chondrocytes for the repair of damaged articulating surfaces. The teachings of the instant invention provide for effective, and efficient means of promoting the transplantation of cartilage and/or chondrocytes into a defect in an articulating joint whereby cartilage is regenerated to fix the defect.

### BRIEF SUMMARY OF THE INVENTION

In one embodiment, the present invention provides an implantable article including a support matrix which can support the growth and attachment of cells thereto, and a method of implanting such an article to regenerate cells at an implantation location. In one embodiment, the present invention provides a method for the effective treatment of articulating joint surface cartilage in an animal by the transplantation of an implantable article including chondrocyte cells retained on an absorbable support matrix.

In one embodiment, chondrocyte cells are retained only on an edge of the matrix. In one embodiment, the support matrix is a covering matrix made from collagen, and the chondrocyte cells are autologous or homologous. In another embodiment, the support matrix is made from collagen and elastin, or collagen and one or more other resorbable materials. In another embodiment, the support matrix is made from small intestine submucosa from animal sources.

In another embodiment, the support matrix is made from pericardium. In a different embodiment, the support matrix is made from collagen and one or more other materials related to polyesters.

The implantable article preferably is secured to the transplantation site by an adhesive or mechanical retention means. The present invention also is directed to an instrument for placing and manipulating the implantable article at the site of implantation, and a retention device for securing the implantable article to the site of implantation.

The present invention is also directed to an implantable article for cartilage repair in an animal, the implantable article including chondrocyte cells retained to an absorbable support matrix, and a method of making same.

In another embodiment, the present invention is directed to a method for treating an articulating joint surface cartilage including the placing chrondrocytes upon a surface to be treated, and covering the surface to be treated with a covering matrix. The covering matrix is secured to the area of cartilage surrounding the defect.

### BRIEF DESCRIPTION OF THE DRAWINGS

FIG. 1A is a drawing showing a typical articulating end of a bone. Typically, the bone material is covered on the articulating surface with a cartilaginous cap (shown by stripping labeled cartilage). Where a defect or injury to the cartilaginous cap occurs (Gap in cartilage cap of FIG. 1B), the defective site can be treated directly, or enlarged slightly by surgical procedures. Optionally, a hemostatic barrier (numbered 1) may be placed within the defect in the cartilage cap to inhibit or prevent vascularization into regenerating cartilage from the underlying bone (FIG. 1C), when necessary, for example in defects that extend into or below the subchondral layer. The chondrocytes to be implanted into the defect cavity are then layered on top of this hemostatic barrier, or directly on top of the defect.

FIG. 2 is a drawing showing the treated defect (gap in stippling area) in the cartilaginous cap (stippling area) covered by a matrix which is used to form a cap/patch or bandage over the defect site. This cap is fixed in place, either sutured or glued to the edge of the cavity to healthy cartilage or otherwise attached. This cap is covering the defective area of the joint into which the cultured chondrocytes have been transplanted.

FIG. 3A shows a typical articulating end of a bone in a knee joint, having an articulating surface with a cartilaginous cap.

FIG. 3B shows a cartilage defect or injury to a cartilaginous cap of an articulating end of a bone.

FIG. 4 shows one embodiment of an implantable article according to the present invention.

FIG. 5 shows how the implantable article of FIG. 4 may be disposed for implantation in an arthroscopic introducer such as that shown in FIG. 6.

FIG. 6 shows an arthroscopic introducer for implanting the implantable article at the site of implantation, according to the present invention.

FIG. 7 is a drawing schematically illustrating the placement of the implantable article of FIG. 5 at the site of defect or injury in the cartilaginous cap using two access channels which can accommodate arthroscopic instruments.

FIG. 8 is a schematic cross section of cartilage with a defect or injury which does not extend into the subchondral layer, and an implantable article according to the present invention secured by adhesive to the site of defect or injury.

FIG. 9 is a schematic cross section of cartilage with a defect or injury which does not extend into the subchondral layer, and an implantable article secured to the site of defect or injury by a mechanical retainer.

FIG. 10 illustrates one embodiment of the mechanical retainer used to secure the implantable article to the site of defect or injury.

FIG. 11 is a schematic cross section of cartilage with a defect or injury which extends into the subchondral layer, and an implantable article according to the present invention secured by adhesive to the site of defect or injury.

FIG. 12 is a schematic cross section of cartilage with a defect or injury which extends into the subchondral layer, and an implantable article secured to the site of defect or injury by a mechanical retainer.

FIG. 13A is a black and white copy of a color microphotograph of histological specimen of a solid support matrix at the beginning of chondrocyte cell growth thereon.

FIG. 13AA is the color microphotograph of FIG. 13A.

FIG. 13B is a black and white copy of a color microphotograph showing the support matrix of FIG. 13A loaded with chondrocyte cells after three weeks of chondrocyte cell growth thereon.

FIG. 13BB is the color microphotograph of FIG. 13B.

FIG. 13C is a photograph showing a support matrix formed of collagen having chondrocyte cells grown thereon, shown by immunohistochemical staining.

FIG. 13D is a photograph showing a support matrix formed of collagen, and having chondrocyte cells grown thereon in a bioreactor system, shown by immunohistochemical staining.

FIG. 14 is a photomicrograph showing chondrocyte cells 24 on the DePuy support matrix 22.

FIG. 15 is a graph depicting total cell numbers in control (black shaded), Chondro-Gide membrane group (stippled), and DePuy membrane group (gray shaded) at 3 days, 2 weeks, and 6 weeks.

FIG. 16 is a graph depicting cell viability in control (black shaded), Chondro-Gide membrane group (stippled), and DePuy membrane group (gray shaded) at 3 days, 2 weeks, and 6 weeks.

### DETAILED DESCRIPTION OF THE INVENTION

In one embodiment, the present invention concerns the use of certain products that inhibit the formation of vascular tissue, for instance such as capillary loops projecting into the cartilage being established, during the process of autologous transplantation of chondrocytes into defects in the cartilage. Such products are useful in repairing cartilage defects in bones where the defects extend into or below the subchondral layer, sometimes referred to as a full thickness defect. The formation of vascular tissue from the underlying bone will tend to project into the new cartilage to be formed leading to the appearance of cells other than the mesenchymal specialized chondrocytes desired.

The contaminating cells introduced by the vascularization may give rise to encroachment and over-growth into the cartilage to be formed by the implanted chondrocytes. One of the types of commercial products which can be used in this invention is Surgicel® (Ethicon Ltd., UK) which is absorbable after a period of 7-14 days. This is contrary to the normal use of a hemostatic device, such as Surgicel®, as described in a product insert from Ethicon Ltd.

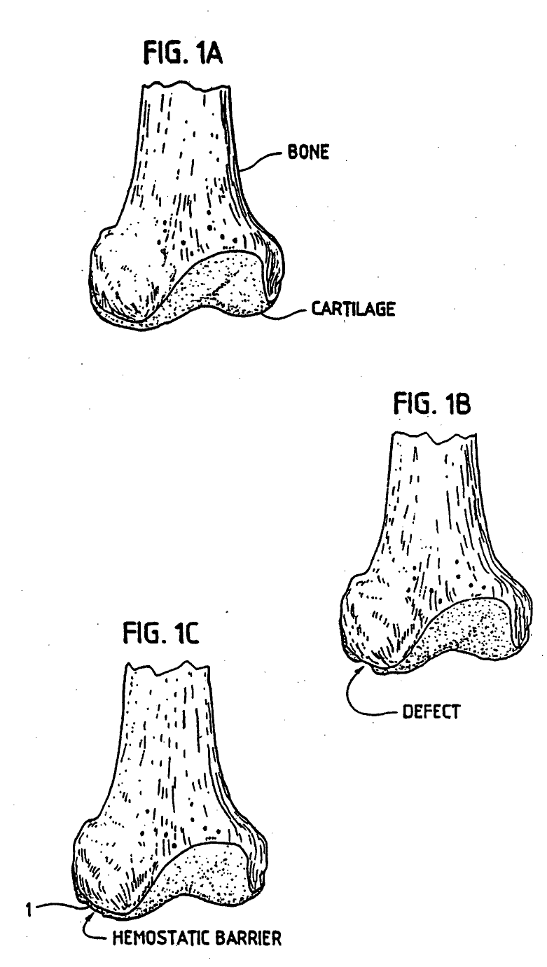

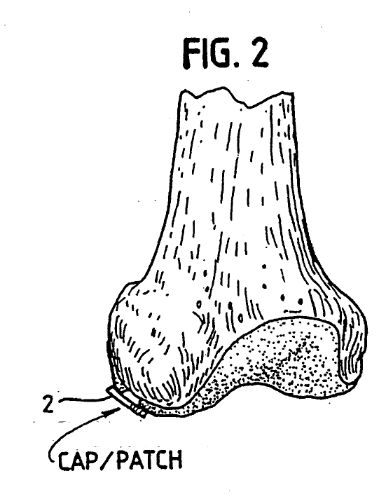

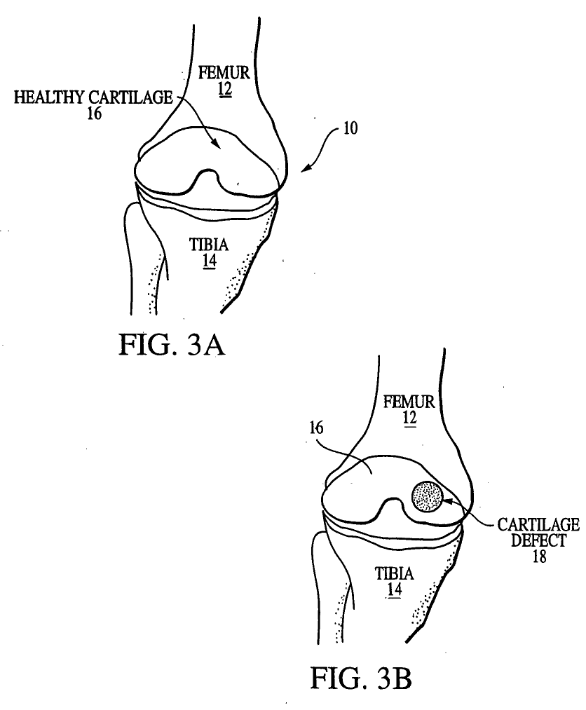

Surprisingly, we have found that in a situation where one desires to inhibit the re-vascularization of cartilage, a hemostatic material can be used and will act as a gel like artificial coagulate. FIG. 1C describes such a hemostatic barrier (numbered 1) that can be used to inhibit the re-vascularization in a cartilage defect. If red blood cells should be present within a full-thickness defect of articular cartilage that is covered by such a hemostatic barrier, these blood cells will be chemically changed to hematin and thus not be able to induce vascular growth. Thus, a hemostatic product used as a re-vascularization inhibitory barrier with or without fibrin adhesives, such as for example Surgicel®, is effective for the envisioned method as taught by the instant invention. In another aspect of this invention, a matrix, such as a cell-free matrix or another matrix described below, is used as a patch covering or inserted into the defective area of the joint into which the cultured chondrocytes are being transplanted using, for example, autologous chondrocytes for the transplantation. Further, the present invention may also utilize allogeneic chondrocytes or xenogeneic chondrocytes for the repair of a cartilage defect. FIG. 2 depicts the covering matrix 2 that can be used as a patch to cover the defect area and FIG. 3B depicts a localization of a cartilage defect.

Thus, in one embodiment the instant invention teaches methods for effective repair or treatment of cartilage defects in articular joint bone surfaces which comprises administering an agent or device to block vascular invasion of the cartilage site to be repaired, and also providing for a matrix which will isolate the repair site and keep transplanted cells in place. Thus the instant invention also provides for a kit, comprising an optional hemostatic component for insertion into the site to be repaired such that there is an effective inhibition of vascularization into the site to be repaired; and once the chondrocytes to be transplanted are placed into the site to be repaired, a matrix 2 is capped over the repair site such that the transplanted chondrocytes are held in place, but are still able to gain access to nutrients.

Certain aspects of the invention have been exemplified using an in vitro system to study the behavior of the chondrocytes when in contact with a certain product or a combination of certain products that will inhibit the formation of vascular tissue. This in vitro testing predicts the ability of certain tested materials to support chondrocyte cell growth thereon, tests matrices for use as implants having chrondrocytes held thereon, and tests each matrix's ability to inhibit vascularization, as will occur in vivo where capillary loops project into the cartilage being established during the process of autologous transplantation of chondrocytes into defects in the cartilage.

Suitable hemostatic products will be characterized by having the ability to inhibit the growth, or invasion of vascular tissue, osteocytes, fibroblasts, etc. into the developing cartilage. A suitable hemostatic material will achieve the goal of the methods of the instant invention in that vascular and cellular invasion into developing cartilage should be prevented in order to optimize the formation of cartilage and achieve repair of the full thickness of any defects in the articular cartilage. Ideally, the hemostatic barrier will be stable for an extended period of time sufficient to allow for full cartilage repair, and then be able to be absorbed or otherwise broken down over time. One material identified as suitable is called Surgicel® W1912 (Lot GG3DH, Ethicon Ltd. UK), and is an absorbable hemostat such as oxidized regenerated sterile cellulose.

The present invention also includes a cartilage repair implant and implantation method and apparatus for such an implant. The implant comprises a support matrix and autologous or homologous chondrocyte cells retained thereon. In one embodiment, the chondrocyte cells are retained only on one or more edges or layers of the matrix. Generally, the support matrix is a material which will support chondrocyte cell growth and which, over time will be absorbed in a body of a patient receiving the implant. The transplantation procedure may be by arthroscopic, minimally invasive or open surgery techniques. The method of the invention also contemplates the use of suitable allogenic and xenogenic chondrocyte cells for the repair of a cartilage defect.

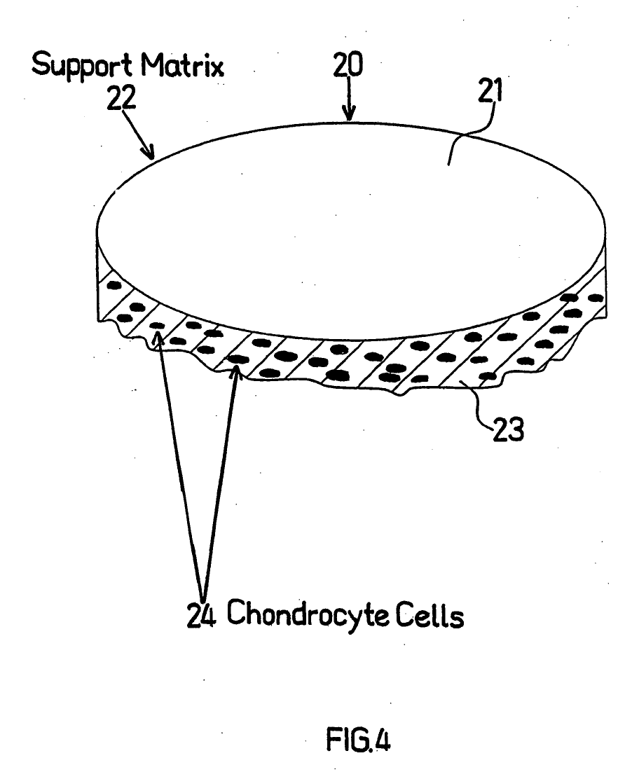

FIG. 4 shows such an implant. More specifically, an implantable article 20 includes a support matrix 22 having chondrocyte cells 24 retained thereon. A suitable support matrix 22 will be a solid or gel-like scaffold characterized by being able to hold a stable form for a period of time to enable the growth of chondrocytes cells thereon, both before transplant and after transplant, and to provide a system similar to the natural environment of the chondrocyte cells to optimize chondrocyte cell growth differentiation.

Support matrix 22 will be stable for a period of time sufficient to allow full cartilage repair and then be absorbed by the body over time, for example, within two to three months without leaving any significant traces and without forming toxic degradation products. The term “absorbed” is meant to include processes by which the support matrix is broken down by natural biological processes, and the broken down support matrix and degradation products thereof are disposed of, for example, through the lymphatics or blood vessels. Accordingly, support matrix 22 preferably is a physiologically absorbable, nonantigenic membrane-like material. Further, in one embodiment support matrix 22 preferably is in a sheet like form having one relatively smooth side 21 and one relatively rough side 23. Rough side 23 typically faces cartilage defect 18 and promotes chondrocyte cell ingrowth, while the smooth side 21 typically faces away from cartilage defect 18 and impedes tissue ingrowth. In another embodiment, support matrix 22 has two smooth sides of similar porosity.

In one embodiment, support matrix 22 is formed of polypeptides or proteins. Preferably, the polypeptides or proteins are obtained from natural sources, e.g., from mammals. Artificial materials, however, having physical and chemical properties comparable to polypeptides or proteins from natural sources, may also be used to form support matrix 22. It is also preferred that support matrix 22 is reversibly deformable as it is handled by the user so implantable article 20 can be manipulated and then returns to its original shape as described below, during one aspect of the present invention.

A preferred material from which support matrix 22 is formed is collagen such as that obtained from equine, porcine, bovine, ovine, and chicken. Suitable materials from which support matrix 22 can be formed include ChondroCell® (a commercially available type II collagen matrix pad, Ed. Geistlich Sohne, Switzerland), and Chondro-Gide® (a commercially available type I collagen matrix pad, Ed. Geistlich Sohne, Switzerland). A support matrix 22 formed of collagen Type I is somewhat stiffer than a support matrix formed from collagen Type II, although Type II collagen matrices may also be used. Another preferred material from which support matrix is formed is a cross-linked or uncross-linked form of Permacol™ (Tissue Science Laboratories, UK).

Alternatively, collagen is obtained from a marine sponge as described in Swatschek, et al., 2002, Eur. J. of Pharmaceutics and Biopharmaceutics, 53:107-113, and in Australian patent application number AU 3741701. Briefly, collagen is isolated from a marine sponge by washing the sponge several times in water, mincing and homogenizing the sponge tissue, treating the solution with a high concentration of urea, centrifuging the resulting solution, and precipitating the collagen from the supernatant.

An implantable article as described above may be made, for example, by culturing chondrocyte cells on this support matrix as described in more detail below.

For an autologous implant, a cartilage biopsy first is harvested by arthroscopic technique from a non-weight bearing area in a joint of the patient and transported to the laboratory in a growth media containing 20% fetal calf serum.

The cartilage biopsy is then treated with an enzyme such as trypsin ethylenediaminetetraacetic acid (EDTA), a proteolytic enzyme and binding agent, to isolate and extract cartilage chondrocyte cells. The extracted chondrocyte cells are then cultured in the growth media from an initial cell count of about 50,000 cells to a final count of about 20 million chondrocyte cells or more.

Three (3) days before re-implantation, the growth media is exchanged for a transplant media which contains 10% autologous serum (that is, serum extracted from the patient's blood as described below). Then, the cultured chondrocyte cells in the transplant media are soaked into and penetrate one or more layers of support matrix 22, and continue multiplying to form implantable article 22. Preferably, chondrocyte cells are adhered only to one edge or an outer layer of support matrix 22. Implantable article 22 is then implanted at a site of cartilage defect 18 in the patient.

It is understood that defect or injury 18 can be treated directly, enlarged slightly, or sculpted by surgical procedure prior to implant such as described in U.S. patent application Ser. No. 09/320,246, to accommodate implantable article 20. The culturing procedure as well as the growth and transplant medias are described by way of example, in detail below, starting first with a description of a laboratory procedure used to process the harvested cartilage biopsy and to culture the chondrocyte cells according to the present invention.

Growth media (hereinafter, “the growth media”) used to treat the cartilage biopsy during the culturing process and to grow the cartilage chondrocyte cells is prepared by mixing together 2.5 ml gentomycin sulfate (concentration 70 micromole/liter), 4.0 ml amphotericin (concentration 2.2 micromole/liter; tradename Fungizone®, an antifungal available from Squibb), 15 ml 1-ascorbic acid (300 micromole/liter), 100 ml fetal calf serum (final concentration 20%), and the remainder DMEM/F12 media to produce about 400 ml of growth media. (The same growth media is also used to transport the cartilage biopsy from the hospital to the laboratory in which the chondrocyte cells are extracted and multiplied.)

Blood obtained from the patient is centrifuged at approximately 3,000 rpm to separate the blood serum from other blood constituents. The separated blood serum is saved and used at a later stage of the culturing process and transplant procedure.

Cartilage biopsy previously harvested from a patient for autologous transplantation is shipped in the growth media described above to the laboratory where it will be cultured. The growth media is decanted to separate out the cartilage biopsy, and discarded upon arrival at the laboratory. The cartilage biopsy is then washed in plain DMEM/F12 at least three times to remove the film of fetal calf serum on the cartilage biopsy.

The cartilage biopsy is then washed in a composition which includes the growth media described above, to which 28 ml trypsin EDTA (concentration 0.05%) has been added. In this composition it is incubated for five to ten minutes at 37° C., and 5% CO₂. After incubation, the cartilage biopsy is washed two to three times in the growth media, to cleanse the biopsy of any of the trypsin enzyme. The cartilage is then weighed. Typically, the minimum amount of cartilage required to grow cartilage chondrocyte cells is about 80-100 mg. A somewhat larger amount, such as 200 to 300 mg, is preferred. After weighing, the cartilage is placed in a mixture of 2 ml collagenase (concentration 5,000 enzymatic units; a digestive enzyme) in approximately 50 ml plain DMEM/F12 media, and minced to allow the enzyme to partially digest the cartilage. After mincing, the minced cartilage is transferred into a bottle using a funnel, and approximately 50 ml of the collagenase and plain DMEM/F12 mixture is added to the bottle. The minced cartilage is then incubated for 17 to 21 hours at 37° C., and 5% CO₂.

In one embodiment, the incubated minced cartilage is then strained using a 40 μm mesh, centrifuged (at 1054 rpm, or 200 times gravity) for 10 minutes, and washed twice with growth media. The chondrocyte cells are then counted to determine their viability, following which the chondrocyte cells are incubated in the growth media for at least two weeks at 37° C., and 5% CO₂, during which time the growth media was changed three to four times.

At least three days before re-implantation in the patient, the chondrocyte cells are removed by trypsinization and centrifugation from the growth media, and transferred to a transplant media containing 1.25 ml gentamycin sulfate (concentration 70 micromole/liter), 2.0 ml amphotericin (concentration 2.2 micromole/liter; tradename Fungizone®, an antifungal available from Squibb), 7.5 ml 1-ascorbic acid (300 micromole/liter), 25 ml autologous blood serum (final concentration 10%), and the remainder DMEM/F12 media to produce about 300 ml of transplant media.

Support matrix 22 is then cut to a suitable size fitting into the bottom of a well in a NUNCLON™ cell culture tray, and then placed under aseptic conditions on the bottom of the well with 1-2 ml transplant media. A sufficient number of cultivated cartilage chondrocyte cells (e.g. 3-10 million chondrocyte cells) in approximately 5-10 ml of the transplant media, are then soaked into support matrix 22, and incubated approximately 72 hours at 37° C., and 5% CO₂ to allow the chondrocyte cells to continue to grow. During this incubation, the chondrocyte cells arrange in clusters and adhere to support matrix 22. In one embodiment, the chondrocyte cells are adhered to only one outer layer of one side of support matrix 22. Using this method, it has been found that support matrix 22 supports the growth and retention of chondrocyte cells thereon in a sufficient number to form implantable article 20, without significant loss of the biomechanical properties of support matrix 22. Support matrix 22 also provides an environment to support continued growth of chondrocyte cells after implantation of the implantable article at the site of the cartilage defect.

In another embodiment, following the 17-21 hour incubation period and after determining cell count and viability as discussed above, the chondrocyte cells are transferred to the transplant media and then grown directly on support matrix 22 as described above for a period of at least two weeks.

It has been found that implantable article 20 temporarily can be deformed without mechanical destruction or loss of the chondrocyte cells adhered to support matrix 22. This deformation is completely reversible once implantable article 20 is introduced into the joint or is placed on the surface to be treated, as described below.

Accordingly, and in accordance with another aspect of the present invention, support matrix 22 onto which chondrocyte cells are grown or loaded in a sufficient number, temporarily can be deformed in a way that allows its introduction into the working device of an arthroscope without mechanical destruction or loss of its chondrocyte cell load.

At the same time it has been found that this matrix can be secured by adhesive or mechanical retention means, to the cartilage defect area without impairing the further in situ differentiation of the chondrocytes and the regeneration of the natural cartilage matrix material.

Other aspects of the present invention include instruments to place implantable article 20 at the implantation site, and a mechanical retention device to hold implantable article 20 at the implantation site.

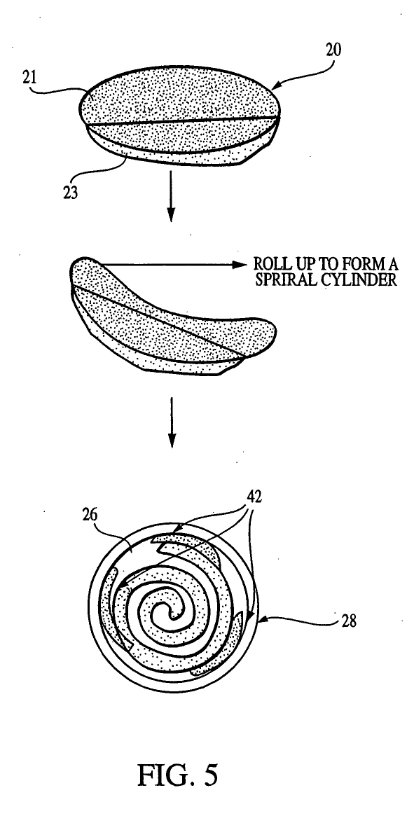

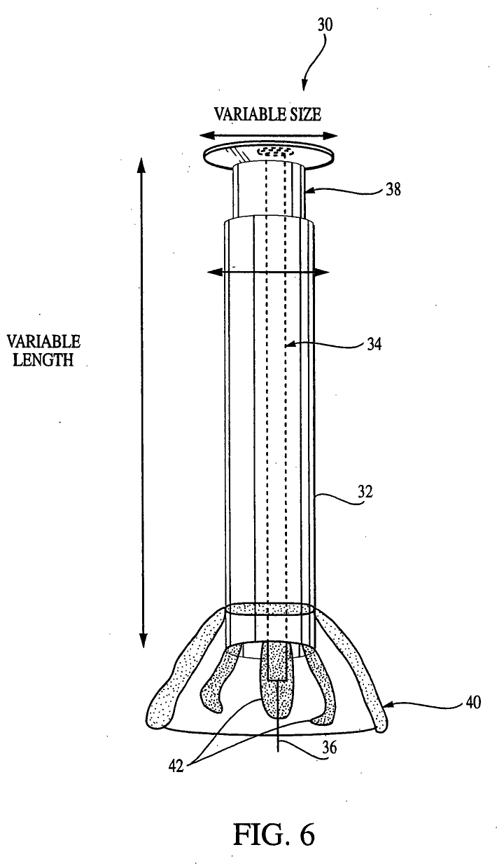

In one embodiment of the present invention, the implantation procedure is performed by an arthroscopic technique. FIG. 5 shows how implantable article 20 can be rolled across the diameter thereof to form a spirally cylindrical transplant so that implantable article 20 can be delivered to an implantation site through a working channel 26 of an arthroscopic introducer 28. A suitable arthroscopic introducer is depicted in FIG. 6.

In FIG. 6, an arthroscopic introducer 30 includes a working channel 32 having a diameter and length suitable to enter the joint of interest and to deliver the desired dimension of implantable article 20. For example, for most procedures, the diameter of working channel 32 is approximately 8-20 mm, and the length is approximately 30-60 cm. Within and longitudinally movable with respect to working channel 32 is an injection channel 34 accommodating a retractable and removable needle 36. Injection channel 32 is attached to a handle 38 which is telescopically depressible at least partially into working channel 32. Needle 36 extends the length of injection channel 34 and allows fluids to pass therethrough to the site of implantation. Injection channel 34 is moved within working channel 32 by telescopically moving handle 38 toward or away from the implantation site.

Introducer 30 also includes a cap 40 made of rubber or other suitable material, slideably engaged on introducer 30. In use, cap 40 surrounds the site of the cartilage defect and excludes fluids, such as blood and other natural fluids, from flowing into the site of the cartilage defect. Introducer 30 also has two or more outwardly biased gripping elements 42 attached to handle 38, for grasping, introducing and placing implantable article 20 at the implantation site. In use, as handle 38 is telescopically moved toward and away from the user, gripping elements 42 engage the inside of working channel 32 and are moved toward each other in a gripping manner (as handle 38 is moved toward the user), and away from each other to release the grip (as handle 38 is moved away from the user). Such telescopic movement may be controlled by a biasing element (not shown) disposed within handle 38 which allows injection channel 34 and gripping elements 42 to be slideably advanced and retracted within working channel 42.

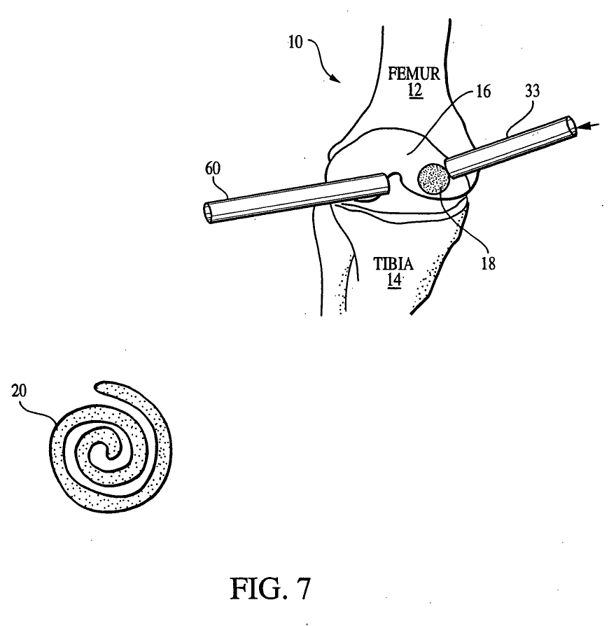

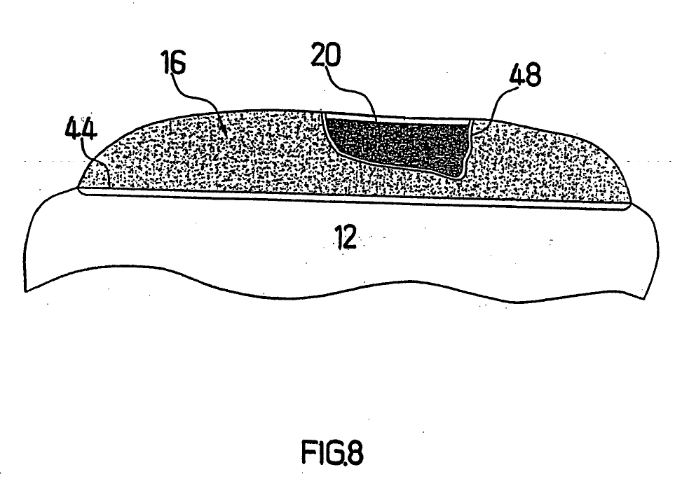

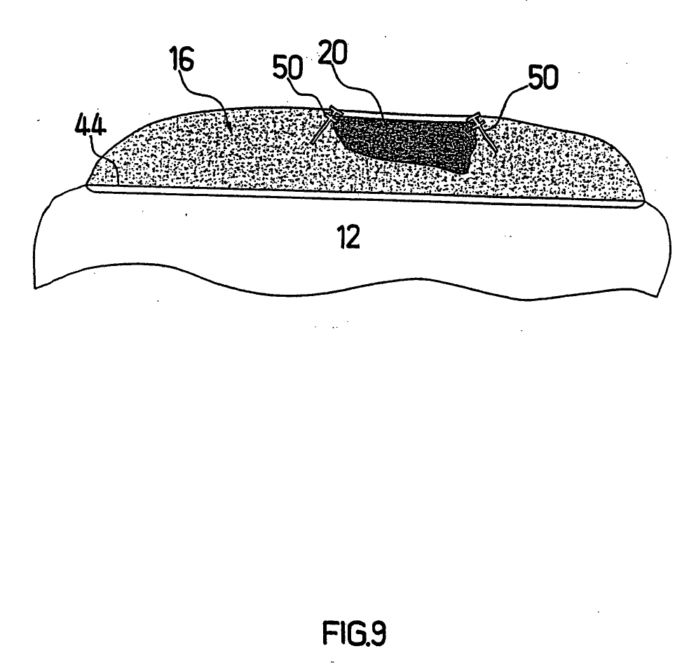

FIGS. 7-9 show a typical arthroscopic procedure for implanting implantable article 22 at a site of implantation such as knee joint 10. Defective cartilage 18 is removed from the site of the defect, preferably to a depth above subchondral layer 44 leaving a well 46 (See FIGS. 8-9). After cartilage defect 18 is removed, the defect site is prepared to receive implantable article 22. If the subchondral layer has been disturbed to the point that bleeding occurs at the implantation site, the site optionally may first be covered with any absorbable material which acts as a hemostatic barrier.

Otherwise, site preparation may include injection of a biocompatible glue through needle 36 into well 46. Such a biocompatible glue, seen as adhesive 48 in FIG. 8, may comprise an organic fibrin glue (e.g., Tisseel®, fibrin based adhesive, Baxter, Austria or a fibrin glue prepared in the surgical theater using autologous blood samples).

Implantable article 20 previously cut to the desired dimension, and rolled into a spiral cylindrical shape as shown in FIG. 7 is then gripped by gripping elements 42 and held within the end of arthroscopic introducer 30. Arthroscopic introducer 30 holding implantable article 20 within its end, is then advanced to the site of implantation through an access channel 33, released from gripping elements 42, and unrolled using gripping elements 42 or allowed to unroll as it exits working channel 32. Access channel 33 includes one or more channels that allow instruments such as introducer 30 and visualization instruments, to access the transplantation site. Using gripping elements 42, implantable article 20 is manipulated such that the side holding chondrocytes therein, in this embodiment rough side 23 of implantable article 20, faces well 46 and is gently held in place in well 46 to allow adhesive 48 to harden and bind implantable article 20 in well 46.

As shown in FIG. 8, a second access channel having one or more channels may be used to allow access of instruments to the site of implantation to assist in placement of the implantable article, adhesive and/or mechanical retention means, or to allow for access of visualization instruments to the site of implantation. Such a separate access channel may also be used to perform one or more of the functions described in relation to arthroscopic introducer 30 or other arthroscopic instruments.

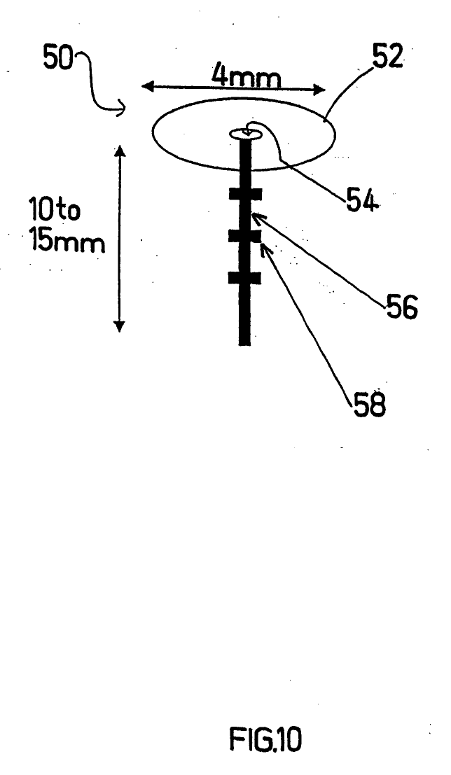

In another embodiment (FIG. 9), mechanical retention means such as absorbable pins, anchors, screws or sutures are used to secure implantable article 20 in well 46. Suitable pins 50 include Ortho-Pin™ (a commercially available lactide co-polymer pin, Ed. Geistlich Sohne, Switzerland). FIG. 10 shows one embodiment of absorbable pin 50. In this embodiment, pin 50 includes head 52, intramedullar channel 54 within shaft 56, and one or more retention rings 58. The dimensions of pin 50 will vary with the particular use, but, typically, pin 50 is about 10-15 mm in length, head 52 is about 4 mm in diameter, intramedullar channel 54 is approximately 1.2 mm in diameter, shaft 56 is approximately 2 mm in diameter, and retention rings 58 are about 2.5 mm in diameter. Retention rings 58 serve to anchor pin 50 into healthy cartilage surrounding the cartilage defect. Pin 50 is formed from any material that will not harm the body and can be absorbed or otherwise broken down by the body after a period of time. For example, pin 50 may be made from polylactide.

It is also contemplated to be within the scope of the present invention to use a combination of adhesive 48 and mechanical retention means such as pins 50 to secure implantable article 20 in well 46.

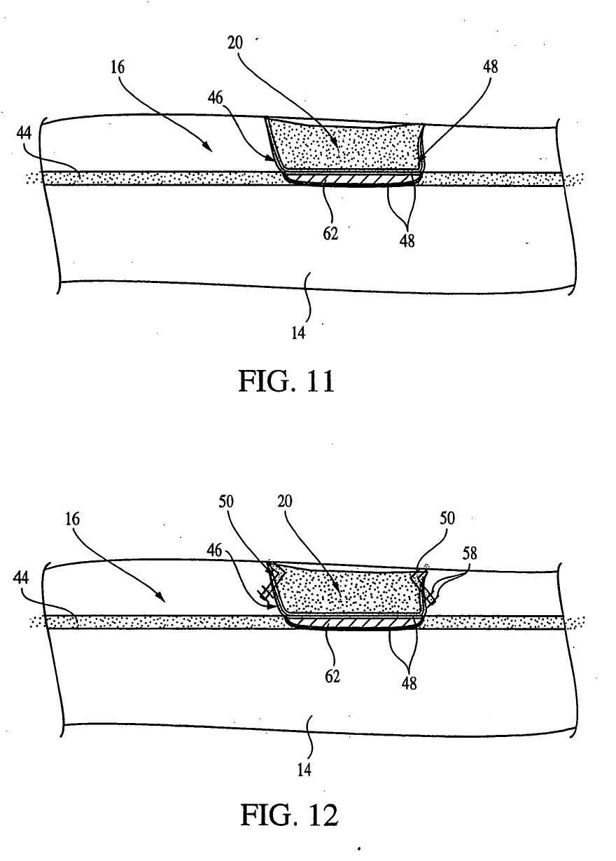

As indicated above, where cartilage defect 18 extends into or below subchondral layer 44, or requires removal of cartilage into or below subchondral layer 44 as shown in FIGS. 11 and 12, the above procedure is modified optionally to include placement of a hemostatic barrier 62 in well 46 prior to placement of implantable article 20. With such defects, a physician optionally can use hemostatic barrier 62 to inhibit the growth or invasion of vascular tissue, osteocytes, fibroblasts, etc. into developing cartilage. This is believed to allow hyaline cartilage to grow at the transplantation site. Suitable hemostatic barriers will inhibit vascularization and cellular invasion into the developing cartilage to optimize formation of cartilage and to achieve growth of the full thickness of cartilage at the defect site. Preferably, the hemostatic barrier is stable for an extended period of time to allow full cartilage repair, and then will be absorbed or otherwise broken down by the body over time. A suitable hemostatic barrier is Surgicel® W1912 (Ethicon, Ltd., United Kingdom), an absorbable hemostat formed of oxidized regenerated sterile cellulose.

The above described surgical instruments are manufactured from any material, such as metal and/or plastic or silicone, suitable for making disposable or multi-use reusable surgical instruments.

Support matrix 22 or covering matrix 2 of the present invention is formed of a collagen membrane, as manufactured by the process described in U.S. Pat. No. 5,028,695 (assigned to Chemokol Gesellschaft Zur Entwicklung von Kollagenprodukten), which is hereby incorporated by reference. The collagen membrane can be prepared from collagen raw material from cattle or pig as follows: the collagen raw material is freed of fatty acid residues, washed with water, treated with an alkali, washed with water, treated with an acid, washed with water, treated again with a strong alkali, treated with an acid, which causes swelling, treated with an inorganic salt to cause shrinkage, the material is squeezed off to a dry weight of 40-50% by weight, the water retained within the material is removed by the addition of a solvent, the material can be cross-linked if necessary, and then dried in stretched form.

Support matrix 22 or covering matrix 2 can also be formed of a membrane including collagen, such as Type I or Type II collagen, and elastin, such as the membrane described in U.S. Pat. No. 5,397,353 (assigned to Oliver, et al., University of Dundee), which is hereby incorporated by reference. The collagen of the collagen/elastin membrane may be obtained from equine, porcine, bovine, ovine, and chicken sources. In one embodiment, support matrix 22 or covering matrix 2 is a collagen/elastin porcine dermis that undergoes numerous organic extraction stages to remove the fat content of the dermis. Once the fat has been removed, the sections undergo numerous enzymatic extractions to remove all cellular material. Such a product prepared according to U.S. Pat. No. 5,397,353 is presumed to be Permacol™ and various cross-linked versions of Permacol™.

Other membranes similar to Permacol™, such as the Rapi-Seal Patch (Fusion Medical Technologies, Inc., Fremont, Calif.) and the Tissue Repair Patch (Glycar Vascular Inc., Dallas, Tex.), may also be used in the present invention.

The collagen/elastin membrane may also be up to 20% cross-linked with polyisocyanates, preferably hexamethylene diisocyanate (HMDI). The collagen/elastin membrane used as support matrix 22 or covering matrix 2 can be in the form of a sheet having two smooth sides and homogenous pore size and texture. Preferably, the collagen/elastin membrane has the following specifications: a thickness of 0.75 mm, a length of 4.8-5.2 cm, a width of 4.8-5.2 cm, a collagen content of >79% and a fat content of 0.4%. Chondrocyte cells can be cultured on this support as described previously above to form an implantable article. The implantable article can be placed into or over the cartilage defect site.

In another embodiment, support matrix 22 can be formed of a Small Intestine Submucosa (“SIS”). The method of preparing the SIS from a segment of small intestine is detailed in U.S. Pat. No. 4,902,508, which is hereby incorporated by reference. A segment of intestine, preferably harvested from porcine, ovine or bovine species, is first subject to abrasion using a longitudinal wiping motion to remove both the outer layers (particularly the tunica serosa and the tunica muscularis) and the inner layers (at least the luminal portions of the tunica mucosa). Typically, the small intestinal submucosa is rinsed with saline and optionally stored in a hydrated or dehydrated state until used as described below.

A plurality of superimposed layers of intestinal submucosa tissue is then compressed, secured to one another and shaped to provide a multi-layered reconstructive structure, as described in U.S. Pat. Nos. 5,788,625, 5,922,028 and 6,176,880 (all assigned to DePuy Orthopaedics (Warsaw, Ind.)), which are hereby incorporated by reference. The multi-layered structure are provided with a sufficient number of submucosal layers to form a reconstructive tissue graft structure having the desired thickness for the replacement of the endogenous cartilaginous structure.

Other SIS membranes which are useful in the present invention include the Suspend Sling™ from Mentor Corporation (Santa Barbara, Calif.), Staple Strips™ from Glycar Vascular, Inc. (Dallas, Tex.), Surgical Fabrics from Boston Scientific (Natick, Mass.), SurgiSIS™ Sling and SurgiSIS™ Mesh from Cook Biotech, Inc. (West Lafayette, Ind.), SIS Hernia Repair Device from Sentron Medical, Inc. (Cincinnati, Ohio), and the Restore® Soft Tissue Implant from DePuy Orthopaedics.

Another membrane that can be employed for support matrix 22 or covering matrix 2 according to the present invention is the resorbable collagen membrane described in U.S. Pat. No. 5,837,278 (assigned to Ed Geistlich Sohne AG), which is hereby incorporated by reference. This resorbable collagen membrane can be derived from naturally occurring membranes, such as sections of hide with a grain side, tendons, and various animal membranes. This collagen membrane has a fibrous side to promote cell growth thereon and a smooth side to inhibit cell adhesion thereon. The membrane is prepared by treatment with an alkali to saponify fats and degrade alkali sensitive substances, treated with acid to degrade acid sensitive substances, washed, dried, degreased and cross-linked if necessary.

Other collagen membranes that can be used as support matrix 22 or covering matrix 2 according to the present invention include FortaFlex™ (prepared from collagen type I) and GraftPatch® (prepared from cross-linked collagen) from Organogenesis, Inc. (Canton, Mass.). Additionally, Antema®, an equine collagen type I composition from Opicrin S.p.A. (Corlo, ITALY), is also useful in the present invention.

Other membranes suitable for use as support matrix 22 or covering matrix 2 include CollaTec membrane from Colla-Tec, Inc. (Plainsboro, N.J.), Collagraft from NeuColl (Campbell, Calif.), BioMend from Integra Life Sciences Corporation (Plainsboro, N.J.), and BioMend® Absorbable Collagen Membrane from Collagen Matrix, Inc. (Franklin Lakes, N.J.). Biosynthetic Surgical Mesh from Advanced UroSciences, Inc., Brennen Medical, Inc. (St. Paul, Minn.), which is prepared from porcine skin (essentially all collagen) and BIOBAR™ from Col-Bar, Ltd. (Ramat-Hasharon, Israel) are also useful materials for support matrix 22 or covering matrix 2 in the present invention.

Additionally, collagen membranes having fibers arranged at the macromolecular level are also suitable for use as support matrix 22 or covering matrix 2. Such membranes are described, for example, in International Patent Publication Number WO02/09790 to Mediolanum Farmaceutici S.P.A. and Opocrin S.P.A.

In another embodiment, support matrix 22 and/or covering matrix 2 can be formed of collagen fibrils which are cross-linked to each other via a reducing sugar or a derivative of a reducing sugar, for example as disclosed in U.S. Pat. No. 5,955,438 (assigned to ColBar R&D Ltd., Ramat-Hasharon, Israel), which is incorporated herein by reference in its entirety. Such sugars can include, but are not limited to, a ketone or aldehyde mono sugar, ribose, glycerose, threose, erythrose, lyxose, xylose, arabinose, allose, altrose, glucose, mannose, gulose, idose, galactose, talose, or any other diose, triose, tetrose, pentose, hexose, septose, octose, nanose, or decose, and combinations of one or more of the same. Support matrix 22 and/or covering matrix 2 can also include antimicrobial agents which have a therapeutic effect during cartilage regeneration. Such antimicrobials include penicillin, cephalosporins, tetracyclines, streptomycin, gentamicin, sulfonamides, antifungals, such as myconazole, and anti-inflammatories, such as cortisone, and combinations of one or more of the above. Factors having tissue inductive properties, such as fibroblast growth factor, platelet derived growth factors, transforming growth factors, differentiating growth factors, and the like, are also included in support matrix 22.

Other collagen materials useful in the present invention are disclosed in U.S. Pat. Nos. 5,256,418 and 5,993,844 (assigned to Organogenesis, Inc.), U.S. Pat. No. 6,206,931 (assigned to Cook Biotech, Inc.), and U.S. Pat. No. 5,026,381 (assigned to Colla-Tec Inc.), all of which are hereby incorporated by reference and which may pertain to one or more products discussed herein.

Products not yet marketed in the U.S. which may be used as support matrix 22 or covering matrix 2 include MACI-MaixR (Matricel, Herzogenrath, Germany), Bio-Seed C (Biotissue, Miami, Fla.), and VivesCart and PLA/PGA copolymers (IsoTis, Bilthoven, Netherlands).

In another embodiment, support matrix 22 or covering matrix 2 can be formed of a sponge of collagen fibers containing antibacterial substances taurolidine and/or taurultam, as described in EP 446,262 (Geistlich Soehne AG, Wolhusen, Switzerland), which is hereby incorporated by reference. The collagen sponge may be obtained from commercial sources, such as from Pentapharm AG of Basel, Switzerland, from Dr. Otto Suwelak GmgH of Billerbeck, West Germany or from Ed Geistlich Söhne A. G. of Wolhusen, Switzerland. The collagen sponge can also be made by conventional methods. For example, bovine skin may be chemically and mechanically treated to separate the epidermis from the underlying associated fat. The layer can then be treated with mild alkali, followed by treatment with acid, and then washed. A proteolytic enzyme may be used to separate collagen from other proteins and a lipase may be used to remove residual fat. The collagen product can then be treated with an oxidizing agent, homogenized, and lyophilized. The incorporation of the taurolidine or taurultam may be effected prior to lyophilization or by redissoving lyophilized collagen in a solution of the taurolidine or taurultam and relyophilizing.

In another embodiment, support matrix 22 or covering matrix 2 can be formed according to the method for producing porous structures described in WO99/27315 (Heshcel Ingo Dipl Ing, Germany), which is hereby incorporated by reference. The porous structures are formed from a liquid or pasty mixture of substances that have at least partially solidified by cooling the mixture between two interspersed surfaces which can be tempered and have varying temperatures. During solidification an ordered structure is formed. The partially solidified product is then freeze-dried to create a homogeneous porous structure.

Support matrix 22 or covering matrix 2 may also be formed of one or more bioabsorbable polymers, such as collagen, fibrin, laminin and fibronectin, having large interconnected pores according to the method described in U.S. Pat. No. 5,869,080 (assigned to Johnson & Johnson Medical Inc., NJ), which is hereby incorporated by reference. This bioabsorbable polymer can be cross-linked, for example, with hexamethylene diisocyanate (HMDI) prior to freezing.

Support matrix 22 or covering matrix 2 can be formed of a collagen sponge such as Instat™ (Johnson & Johnson) as described in Johnson & Johnson brochure entitled “Instat Collagen Absorbable Hemostat”, September 1985, which is hereby incorporated by reference.

In another embodiment, support matrix 22 or covering matrix 2 can be formed of a bioabsorbable sponge according to the method described in U.S. Pat. No. 5,700,476 (assigned to Johnson & Johnson Medical Inc., NJ), which is hereby incorporated by reference. The sponge comprises a matrix structure and at least one substructure and at least one pharmacologically active agent. The matrix and substructure may be made of bioabsorbable materials such as collagen, laminin, elastin, and fibronectin, among others. Further, the sponge matrix may also comprise one or more proteins or one or more polysaccharides, or mixtures thereof. The pharmacological agent may include an antimicrobial, a cytokine, a growth factor, or an antibody, among others.

In another embodiment, support matrix 22 or covering matrix 2 is formed from collagen fibers as described in WO96/25961 (Geistlich Soehne AG), which is hereby incorporated by reference. The matrix may further contain a hydrogel-like material comprising glycosaminoglycans, such as chondroitin sulphate, keratan sulphate, dermatan sulphate and hyaluronic acid, and growth factors. An example of such a matrix is described in U.S. Pat. No. 5,489,304 to Orgill, et al, hereby incorporated by reference in its entirety.

Support matrix 22 and covering matrix 2 can also be formed of a multi-layer membrane comprising a porous matrix layer predominantly of collagen II and at least one dense barrier layer as described in WO99/19005 (Geistlich Soehne AG) and U.S. Publication No. 2002/0013627 (Geistlich, et al.), which are hereby incorporated by reference. The matrix layer has an open sponge-like texture and the barrier layer has a close relatively impermeable texture. In addition, the matrix layer may further contain glycosaminoglycans, such as hyaluronic acid, chondroitin sulphate, keratan sulphate and dermatan sulphate, among others. The matrix layer may also contain laminin, fibronectin calcium alginate or anchorin II and growth factors. The barrier may be made of collagen I and III or synthetic materials such as polyesters, polyglycolic and polylactic acids homopolymers and copolymers, glycolide and lactide copolymers, polyorthoesters and polycaprolactones.

Examples of membranes incorporating synthetic materials such as polyesters are included in the present invention and are exemplified by Parietex® Mesh and Parietex® Composite Mesh from Sofradim Production (Trevoux, France), SepraMesh™ from Genzyme Corporation (Framingham, Mass.), and Composix™ Mesh from C.R. Bard, Inc. (Murray Hill, N.J.).

In another embodiment of the invention, support matrix 22 and/or covering matrix 2 are formed of pericardium. Examples of membranes formed from pericardium which are useful in the present invention include Tutopatch® from Tutogen Medical, Inc. (Parsipanny, N.J.), Peri-Guard Series of membranes and the BioVascular Sling from BioVascular (St. Paul, Minn.).

Certain aspects of the invention have been exemplified by using an in vitro system to study the behavior of chondrocyte cells when in contact with different support matrices. This in vitro testing predicts the ability of certain materials to mechanically withstand the arthroscopic procedure and also provides information as to chondrocyte cell growing behavior.

These and other aspects of the instant invention may be better understood from the following examples, which are meant to illustrate but not to limit the present invention.

### EXAMPLE 1

In order for the Surgicel® to be used according to our invention in preventing development of blood vessels into autologous implanted cartilage or chondrocytes, we treated the Surgicel® with a fixative, such as glutaric aldehyde; we have chosen 0.6% glutaric aldehyde treatment of the Surgicel® for 1 minute, followed by washings to eliminate glutaric aldehyde residues that may otherwise be toxic to tissue. Alternatively, the Surgicel® was treated with the fibrin adhesive called Tisseel® (Immuno AG, Vienna, Austria)), prior to treatment with glutaric aldehyde as described in example 2. We found that the Surgicel® fixated for instance with a fixative such as glutaric aldehyde, washed with sterile physiological saline (0.9%) and stored in refrigerator, does not dissolve for 1 to 2 months. Generally, Surgicel® is resorbed in a period between 7 and 14 days. This time would be too short, because a longer time is needed for preventing the development of blood vessels or vascularization as such from the bone structure into the implanted cartilage before the implanted chondrocytes have grown into a solid cartilage layer getting its nutrition requirements from the neighboring cartilage. In other words sufficient inhibition of the vascularization is needed for a longer time such as for instance one month. Therefore, the product should not be absorbed significantly prior to that time. On the other hand resorption is needed eventually. Hence, the organic material used as an inhibiting barrier shall have these capabilities, and we have found that the Surgicel® treated in this manner provides that function.

### EXAMPLE 2

The Surgicel® was also coated with an organic glue, and in this case we have used Tisseel® as a glue. This product, together with the Surgicel® produces a useable barrier for our particular purpose. Any other hemostatic or vascular inhibiting barrier could be used. The Tisseel® was mixed as described below. The Surgicel® was then coated with Tisseel® by spraying Surgicel® on both sides until soaked. The Tisseel® (a fibrin glue) was then allowed to solidify at room temperature. Immediately prior to completed solidification, the Surgicel® was then placed in 0.6% glutaric aldehyde for 1 minute and then washed with sterile physiological (0.9%) saline. The pH was then adjusted by PBS and/or with NaOH until the pH was stable at 7.2 to 7.4. Afterwards the thus treated Surgicel® was then washed in tissue culture medium such as minimum essential medium/F12 with 15 mM Hepes buffer.

As mentioned in this example we have used Tisseel® as the fibrin adhesive to coat the Surgicel®. Furthermore the fibrin adhesive or glue may also be applied directly on the bottom of the lesion towards the bone, on which the Surgicel® is glued. The in vitro system used, in lieu of in vivo testing, consisted of a NUNCLON™ Delta 6-well sterile disposable plate for cell research work (NUNC (InterMed) Roskilde, Denmark). Each well measures approximately 4 cm in diameter.

In the invention the fibrin adhesive can be any adhesive which together with the fibrin component will produce a glue that can be tolerated in humans (Ihara, N, et al., Burns Incl. Therm. Inj., 1984, 10:396). The invention also anticipates any other glue component that can be used in lieu of the fibrin adhesive. In this example we used Tisseel® or Tissucol® (Immuno AG, Vienna, Austria). The Tisseel® kit consists of the following components:

Tisseel®, a lyophilized, virus-inactivated Sealer, containing clottable protein, thereof: fibrinogen, Plasmafibronectin (CIG) and Factor XIII, and Plasminogen
Aprotinin Solution (bovine)
Thrombin 4 (bovine)
Thrombin 500 (bovine), and
Calcium Chloride solution

The Tisseel® kit contains a DUPLOJECT® Application System. The fibrin adhesive or the two-component sealant using Tisseel® Kit is combined in the manner according to Immuno AG product insert sheet.

### EXAMPLE 3

Chondrocytes were grown in minimal essential culture medium containing HAM F12 and 15 mM Hepes buffer and 5 to 7.5% autologous serum in a CO₂ incubator at 37° C. and handled in a Class 100 laboratory at Verigen Europe A/S, Symbion Science Park, Copenhagen, Denmark. Other compositions of culture medium may be used for culturing the chondrocytes. The cells were trypsinized using trypsin EDTA for 5 to 10 minutes and counted using Trypan Blue viability staining in a Burker-Turk chamber. The cell count was adjusted to 7.5×10⁵ cells per ml. One NUNCLON™ plate was uncovered in the Class 100 laboratory.

The Surgicel® hemostatic barrier was cut to a suitable size fitting into the bottom of the well in the NUNCLON™ tissue culture tray. In this case a circle, of a size of approximately 4 cm (but could be of any possible size) and placed under aseptic conditions on the bottom in well in a NUNCLON™ tissue culture tray. Delta 6 well sterile disposable plate for cell research work (NUNC (InterMed) Roskilde, Denmark). The hemostatic barrier to be placed on the bottom of the well was pretreated as described in Example 1. This treatment delays the absorption of the Surgicel® significantly. This hemostatic barrier was then washed several times in distilled water and subsequently several times until nonreacted glutaraldehyde was washed out. A small amount of the cell culture medium containing serum was applied to be absorbed into the hemostatic barrier and at the same time keeping the hemostatic barrier wet at the bottom of the well.

A number of approximately 10⁶ cells in 1 ml culture medium were placed directly on top of the hemostatic barrier, dispersed over the surface of the hemostatic barrier, pre-treated with 0.4% glutaraldehyde as described above. The plate was then incubated in a CO₂ incubator at 37° C. for 60 minutes. An amount of 2 to 5 ml of tissue culture medium containing 5 to 7.5% serum was carefully added to the well containing the cells avoiding splashing the cells by holding the pipette tip tangential to the side of the well when expelling the medium. It appeared that the pH of the medium was too low (pH about 6.8). The pH was then adjusted to 7.4 to 7.5. The next day some chondrocytes had started to grow on the hemostatic barrier, arranged in clusters. Some of the cells had died due to the low pH exposure prior to the adjustment of the pH. The plate was incubated for 3 to 7 days with medium change at day 3.

At the end of the incubation period the medium was decanted and cold refrigerated 2.5% glutaraldehyde containing 0.1M sodium salt of dimethylarsinic acid, also called sodium cacodylate, pH is adjusted with HCl to 7.4, was added as fixative for preparation of the cell and supporter (hemostatic barrier) for later preparation for electron microscopy.

### EXAMPLE 4

Chondrocytes were grown in minimal essential culture medium containing HAM F12 and 15 mM Hepes buffer and 5 to 7.5% autologous serum in a CO₂ incubator at 37° C. and handled in a Class 100 laboratory at Verigen Europe A/S, Symbion Science Park, Copenhagen, Denmark. Other compositions of culture medium may be used for culturing the chondrocytes. The cells were trypsinized using trypsin EDTA for 5 to 10 minutes and counted using Trypan Blue viability staining in a Buurker-Turk chamber. The cell count was adjusted to 7.5×10⁵ cells per ml. One NUNCLON™ plate was uncovered in the Class 100 laboratory.

The Surgicel® (for use as a hemostatic barrier) was treated with 0.6% glutaric aldehyde for one minute as described in Example 1, and washed with 0.9% sterile sodium chloride solution or, preferably, with a buffer such as a PBS buffer or the culture medium such as MEM/F12, because pH after the glutaric aldehyde treatment is 6.8 and should preferably be 7.0 to 7.5. The Tisseel® was applied on both side of the Surgicel® using the DUPLOJECT® system, thus coating both sides of the Surgicel®, the patch intended to be used, with fibrin adhesive. The glue is left to dry under aseptic condition for at least 3 to 5 minutes. The “coated” hemostatic barrier was placed on the bottom of the well in a NUNCLON™ Delta 6-well sterile disposable plate for cell research work (NUNC (InterMed) Roskilde, Denmark). A small amount of tissue culture medium containing serum was applied to be absorbed into the hemostatic barrier. A number of approximately 10⁶ cells in 1 ml tissue culture medium containing serum was placed directly on top of the hemostatic barrier, dispersed over the surface of the hemostatic barrier. The plate was then incubated in a CO₂ incubator at 37° C. for 60 minutes. An amount of 2 to 5 ml of tissue culture medium containing 5 to 7.5% serum was carefully added to the well containing the cells avoiding splashing the cells by holding the pipette tip tangential to the side of the well when expelling the medium. After 3 to 6 days microscopic examination showed that the cells were adhering to and growing into the Surgicel® in a satisfactory way suggesting that Surgicel® did not show toxicity to the chondrocytes and that the chondrocytes grew in a satisfactory manner into the Surgicel®.

The plate was incubated for 3 to 7 days with medium change at day 3. At the end of the incubation period the medium was decanted and cold refrigerated 2.5% glutaraldehyde containing 0.1M sodium salt of dimethylarsinic acid, also called sodium cacodylate, pH is adjusted with HCl to 7.4, was added as fixative for preparation of the cell and supporter (hemostatic barrier) for later preparation for electron microscopy.

### EXAMPLE 5

Chondrocytes were grown in minimal essential culture medium containing HAM F12 and 15 mM Hepes buffer and 5 to 7.5% autologous serum in a CO₂ incubator at 37° C. and handled in a Class 100 laboratory at Verigen Europe A/S, Symbion Science Park, Copenhagen, Denmark. The cells were trypsinized using trypsin EDTA for 5 to 10 minutes and counted using Trypan Blue viability staining in a Bürker-Türk chamber. The cell count was adjusted to 7.5×10⁵ to 2×10⁶ cells per ml. One NUNCLON™ plate was uncovered in the Class 100 laboratory.

The Bio-Gide® is a resorbable bilayer membrane which will be used as the patch or bandage covering the defective area of the joint into which the cultured chondrocytes are being transplanted by autologous transplantation. The Bio-Gide® is a pure collagen membrane obtained by standardized, controlled manufacturing processes by E. D. Geistlich Sohne AG, CH-61 10 Wolhusen. The collagen is extracted from veterinary certified pigs and is carefully purified to avoid antigenic reactions, and sterilized in double blisters by gamma-irradiation. The bilayer membrane has a porous surface and a dense surface. The membrane is made of collagen type I and type III without further crosslinking or chemical treatment. The collagen is resorbed within 24 weeks. The membrane retains its structural integrity even when wet and it can be fixed by sutures or nails. The membrane may also be “glued” using fibrin adhesive such as Tisseel® to the neighboring cartilage or tissue either instead of sutures or together with sutures.

The Bio-Gide® was uncovered in a class 100 laboratory and placed under aseptic conditions on the bottom of the wells in a NUNCLON™ tissue culture tray. Delta 6 well sterile disposable plate for cell research work (NUNC (InterMed) Roskilde, Denmark), either with the porous surface of the bilayer membrane facing up or with the dense surface facing up. Approximately 10⁶ cells in 1 ml tissue culture medium containing serum was placed directly on top of the Bio-Gide®, dispersed either over the porous or the dense surface of the Bio-Gide®. The plate was then incubated in a CO₂ incubator at 37° C. for 60 minutes. An amount of 2 to 5 ml of tissue culture medium containing 5 to 7.5% serum was carefully added to the well containing the cells avoiding splashing the cells by holding the pipette tip tangential to the side of the well when expelling the medium.

On day 2 after the chondrocytes were placed in the well containing the Bio-Gide®, the cells were examined in a Nikon Invert microscope. It was noticed that some chondrocytes had adhered to the edge of the Bio-Gide®. It was of course not possible to be able to look through the Bio-Gide® itself using this microscope.

The plate was incubated for 3 to 7 days with medium change at day 3. At the end of the incubation period the medium was decanted and cold refrigerated. A solution 2.5% glutaraldehyde containing 0.1M sodium salt of dimethylarsinic acid, also called sodium cacodylate, with the pH adjusted with HCl to 7.4, was added as fixative for preparation of the cell and the Bio-Gide® supporter with the cells either cultured on the porous surface or the dense surface. The Bio-Gide® patches were then sent for electron microscopy at Department of Pathology, Herlev Hospital, Denmark.

The electron Microscopy showed that the chondrocytes cultured on the dense surface of the Bio-Gide® did not grow into the collagen structure of the Bio-Gide®, whereas the cells cultured on the porous surface did indeed grow into the collagen structure and furthermore, showed presence of proteoglycans and no signs of fibroblast structures. This result showed us that when the collagen patch, as for instance a Bio-Gide® patch, is sewn as a patch covering a cartilage defect the porous surface of the collagen matrix should be facing down towards the defect in which the cultured chondrocytes are to be injected. They will then be able to penetrate the collagen and produce a smooth cartilage surface in line with the intact surface, and in this area a smooth layer of proteoglycans will be built up. Whereas, if the dense surface of the collagen patch is facing down into the defect the chondrocytes to be implanted will not integrate with the collagen, and the cells will not produce the same smooth surface as described above.

### EXAMPLE 6

Chondrocytes were grown in minimal essential culture medium containing HAM F12 and 15 mM Hepes buffer and 5 to 7.5% autologous serum in a CO₂ incubator at 37° C. and handled in a Class 100 laboratory at Verigen Europe A/S, Symbion Science Park, Copenhagen, Denmark. The cells were trypsinized using trypsin EDTA for 5 to 10 minutes and counted using Trypan Blue viability staining in a Bürker-Türk chamber. The cell count was adjusted to 7.5×10⁵ to 2×10⁶ cells per ml. One NUNCLON™ plate was uncovered in the Class 100 laboratory.

The Bio-Gide® used as a resorbable bilayer membrane may also be used together with an organic glue such as Tisseel® with additional, significantly higher content of Aprotinin than normally found in the Tisseel®, as described in the product insert. By increasing the content of Aprotinin to about 25,000 KIU/ml, the resorption of the material will be delayed by weeks instead of the normal time span of days.

To test this feature in vitro, the Tisseel® is applied to the bottom of the well of the NUNCLON™ plate, and allowed to solidify incompletely. A collagen patch such as a Bio-Gide® is then applied over the Tisseel® and glued to the bottom of the well. This combination of Bio-Gide® and Tisseel® is designed to be a hemostatic barrier that will inhibit or prevent development or infiltration of blood vessels into the chondrocyte transplantation area. This hybrid collagen patch can now be used for both as a hemostatic barrier at the bottom of the lesion (most proximal to the surface to be repaired) but also as a support for cartilage formation because the distal surface can be the porous side of the collagen patch and thus encourage infiltration of chondrocytes and cartilage matrix. Thus this hybrid collagen patch can also be used to cover the top of the implant with the collagen porous surface directed towards the implanted chondrocytes and the barrier forming the top. The hybrid collagen patch, with an elevated Aprotinin component may also be used without any organic glue such as Tisseel® and placed within the defect directly, adhering by natural forces. Thus the collagen patch can be used both as the hemostatic barrier, and the cell-free covering of the repair/transplant site, with the porous surfaces of the patches oriented towards the transplanted chondrocytes/cartilage. Another variant would use a collagen patch which consists of Type II collagen (Geistlich Sohne AG, CH-61 10 Wolhusen).

Thus the instant invention provides for a hybrid collagen patch where said patch is a collagen matrix with elevated levels of aprotinin component, preferably about 25,000 KIU/ml, in association with organic matrix glue, where the collagen component is similar to the Bio-Gide® resorbable bilayer material or Type II collagen, and the organic glue is similar to the Tisseel® material. In another embodiment, the hybrid collagen patch does not use any organic glue to adhere to the site of repair.

### EXAMPLE 7

A kit as envisioned, will allow for the convenient practice of the method of the instant invention. In a preferred embodiment, a kit of the invention will provide sterile components suitable for easy use in the surgical environment, and will provide a suitable hemostatic barrier, suitable covering patch, and if needed organic glue. A kit of the invention may also provide sterile, cell-free matrix material for supporting autologous chondrocytes that are to be implanted into an articular joint surface defect. In one embodiment, a kit of the invention contains a Surgicel® hemostatic barrier and a Bio-Gide® covering patch with suitable coating of Tisseel® organic glue, where the Surgicel® and Bio-Gide® have been treated according to the teachings of the invention to increase the time till resorption. In instances where Tisseel® is pre-coated, in one embodiment the Tisseel® is supplemented with additional Aprotinin to increase time of resorption.

In another preferred embodiment, the hemostatic barrier and covering patch are both a semi-permeable collagen matrix which is treated to extend the time of resorption of the material. It is also possible to provide Tisseel® glue in enhanced form as a separate component to be applied as needed because of the inherent variability and unique circumstances every repair/transplantation procedure will encounter.

It will be appreciated by persons skilled in the art that numerous variations and/or modifications may be made to the invention shown in the specific embodiments without departing from the spirit or scope of the invention as broadly described. The present embodiments and examples are, therefore, to be considered in all respects as illustrative and not restrictive.

### EXAMPLE 8

Chondrocyte cells were grown for three weeks in the growth media described above in a CO₂ incubator at 37° C. and handled in a Class 100 laboratory at Verigen Transplantation Service ApS, Copenhagen, DK or at University of Lübeck, Lübeck, Germany. [Note that other compositions of growth media may also be used for culturing the chondrocyte cells.] The cells were trypsinized using trypsin EDTA for 5 to 10 minutes and counted using Trypan Blue viability staining in a Bürker-Türk chamber. The cell count was adjusted to 7.5×10⁵ chondrocyte cells per milliliter. One NUNCLON™ plate was uncovered in the Class 100 laboratory.

A support matrix material, specifically a Chondro-Gide® collagen membrane (identical to Bio-Gide® except Chondro-Gide® has a larger width and length than Bio-Gide®; both available from Ed Geistlich Sohne, Geistlich Pharma AG, Wolhusen, Switzerland), was cut to a suitable size to fit into the bottom of a well in a NUNCLON™ cell culture tray. In this case a circle of a size of approximately 4 cm was placed under aseptic conditions on the bottom of the well.

After three weeks, chondrocyte cells were transferred from the growth media to the transplant media described above, and approximately 5×10⁶ chondrocyte cells in 5 ml transplant media were placed directly on top of the support matrix and dispersed over the surface thereof. The plate was incubated in a CO₂ incubator at 37° C. for 3 days. After this period the chondrocyte cells had arranged in clusters and started to grow on the support matrix, and could not be removed from the support matrix by rinsing it with medium or even by mechanically exerting mild pressure on the matrix.

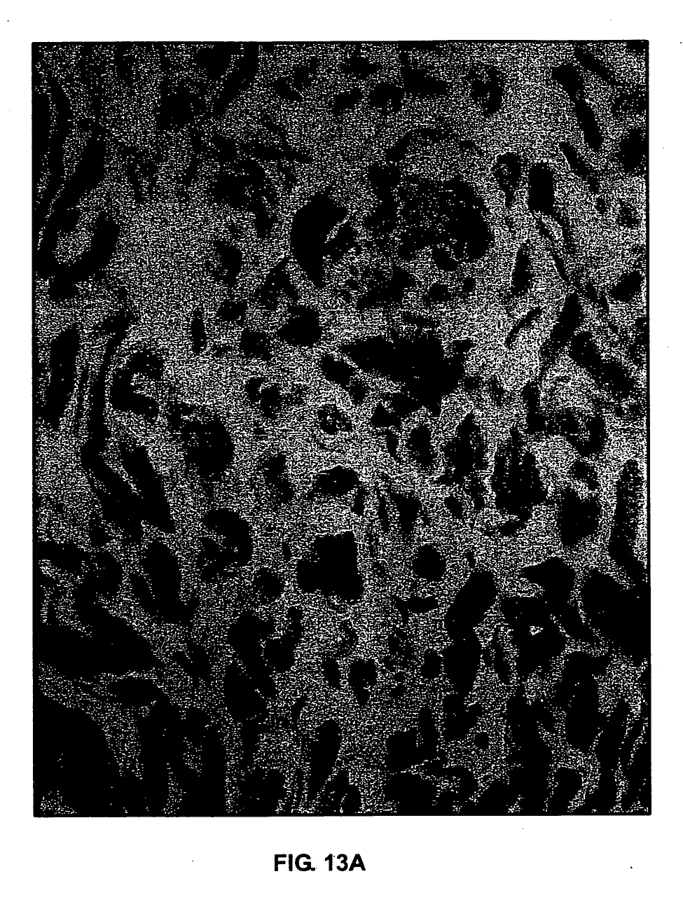

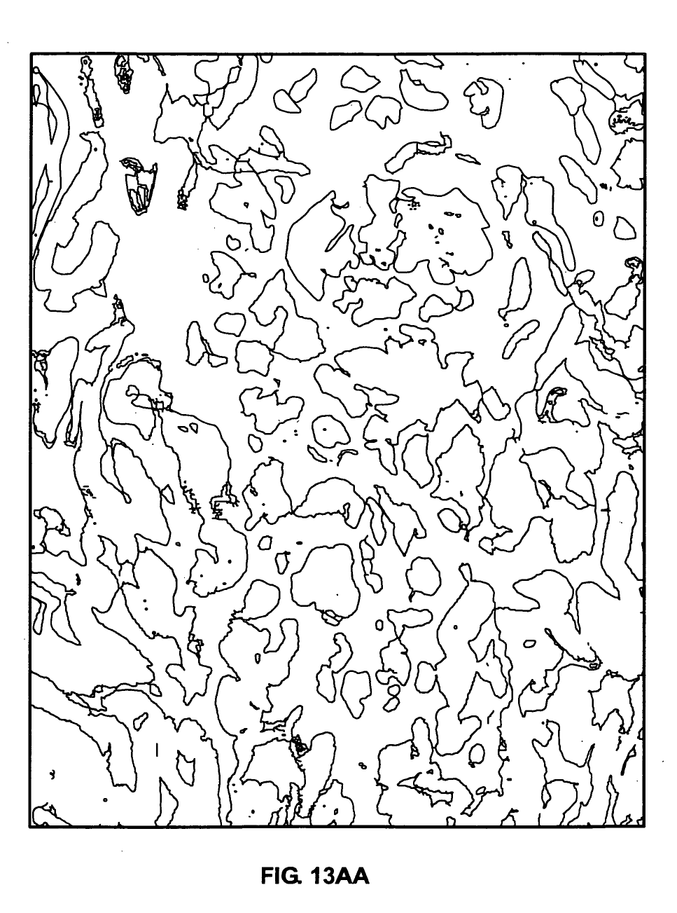

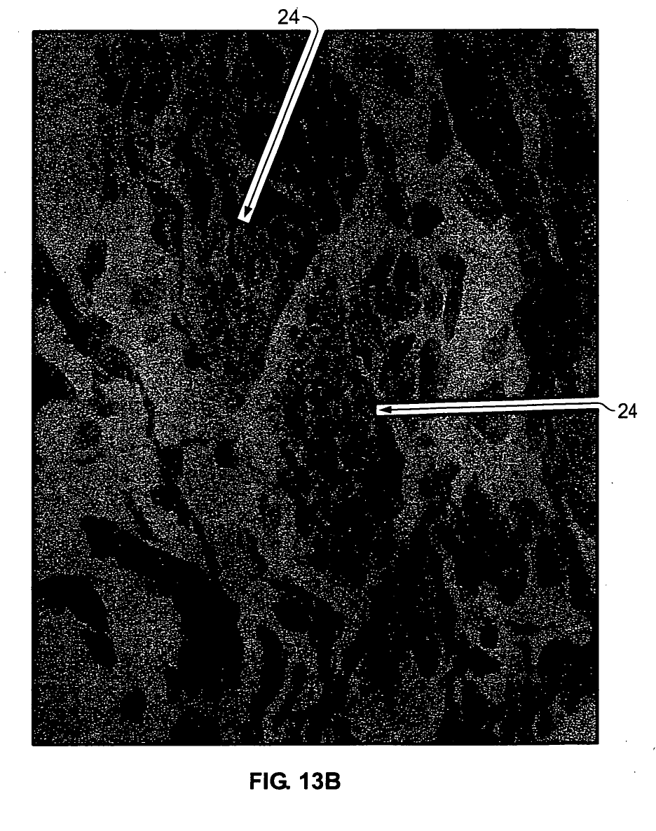

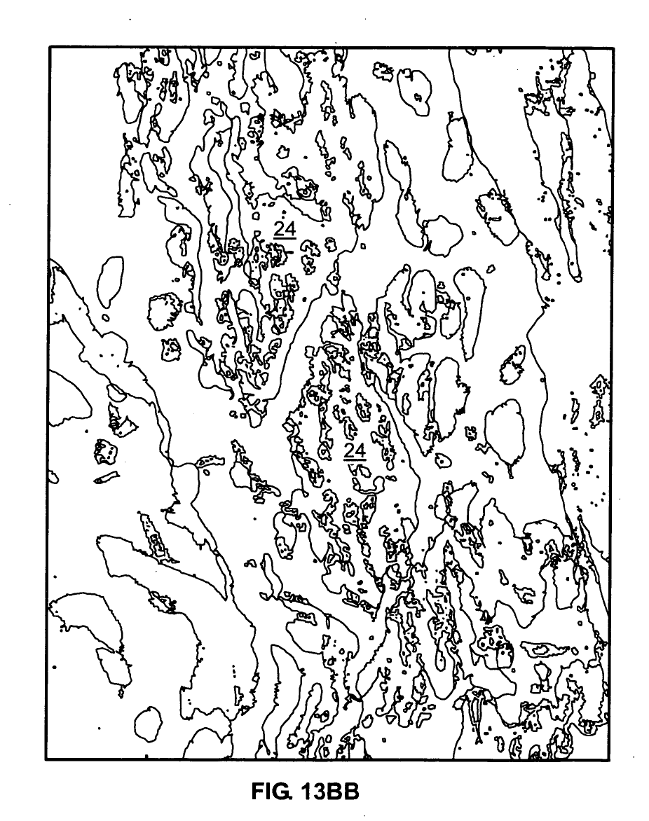

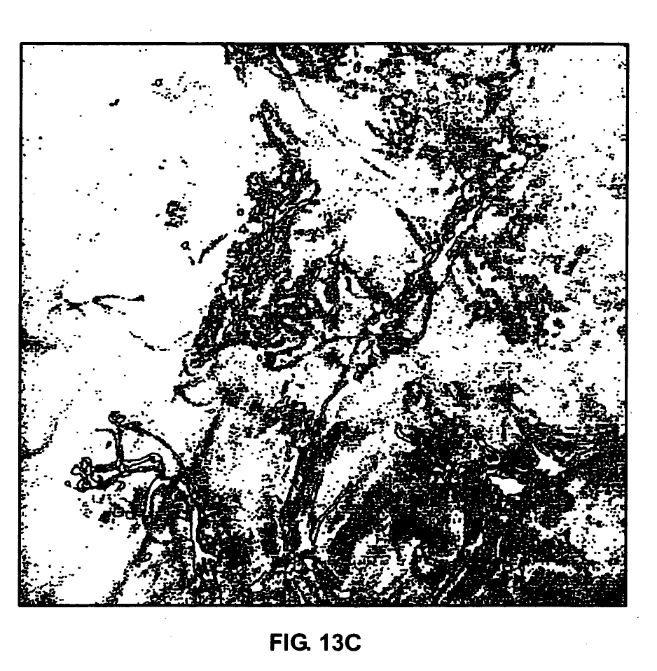

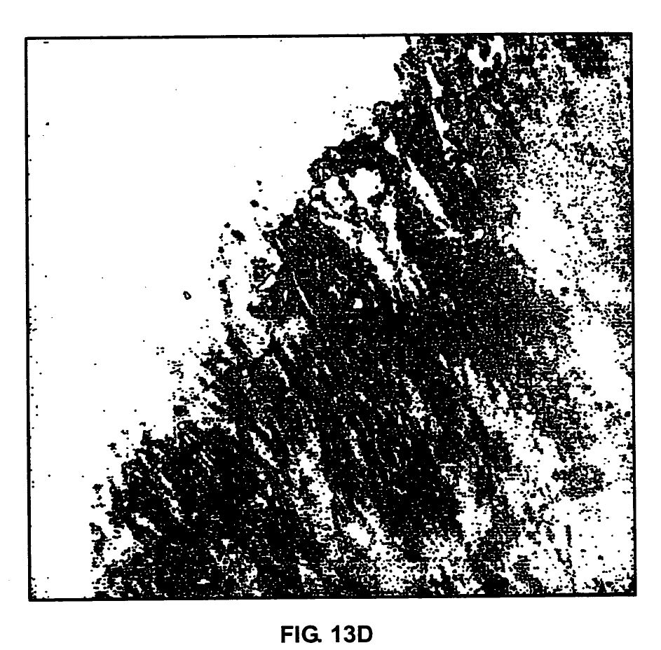

At the end of the incubation period, the transplant media was decanted and the support matrix holding chondrocyte cells grown thereon was cold refrigerated in 2.5% glutaraldehyde containing 0.1 M sodium salt of dimethylarsinic acid, added as fixative. The support matrix was stained with Safranin 0 for histological evaluation. A black and white copy of a color microphotograph thereof is shown in FIG. 13A. A color version of the microphotograph is also submitted as FIG. 13AA to better illustrate the features of the microphotograph.

### EXAMPLE 9

Chondrocytes were grown for three weeks in the growth media described above in a CO₂ incubator at 37° C. and handled in a Class 100 laboratory at Verigen Transplantation Service ApS, Copenhagen, DK or at University of Lübeck, Germany. The cells were trypsinized using trypsin EDTA for 5 to 10 minutes and counted using Trypan Blue viability staining in a Bürker-Turk chamber. The chondrocyte cell count was adjusted to 5×10⁵ chondrocyte cells per milliliter. One NUNCLON™ plate was uncovered in the Class 100 laboratory.

The Chondro-Gide® support matrix, as in Example 1, was cut to a suitable size fitting into the bottom of a well in the NUNCLON™ cell culture tray. In this case a circle of approximately 4 cm in diameter was placed under aseptic conditions on the bottom of the well.

After three weeks, the chondrocyte cells were transferred from the growth media to the transplant media described above, and approximately 5×10⁵ cells in 5 ml transplant media were placed directly on top of the support matrix and dispersed over the surface of the support matrix. The plate was incubated in a CO₂ incubator at 37° C. for 3 weeks.

At the end of the incubation period, the transplant media was decanted, and the support matrix holding the chondrocyte cells thereon was cold refrigerated in 2.5% glutaraldehyde containing 0.1 M sodium salt of dimethylarsinic acid, added as fixative. The support matrix was stained with Safranin 0 for histological evaluation. For immunohistochemistry, collagen membranes were fixed in methanol-acetone and stained for aggrecane and Type II collagen using rabbit anti-human Type II collagen and mouse anti-humane aggrecane. Primary antibodies were visualized using fluorescent secondary antibodies. A black and white copy of a color microphotograph thereof is shown in FIG. 13B showing chondrocyte cells 24. The color version is also submitted as FIG. 13BB to better illustrate the features of the microphotograph.

During the three week incubation period on the Chondro-Gide® support matrix, the chondrocyte cells were observed to have grown and multiplied on the support matrix building clusters in the center of the carrier and lining up along the surface.

### EXAMPLE 10

Chondrocytes were grown for three weeks in the growth media described above in a CO₂ incubator at 37° C. and handled in a Class 100 laboratory at Verigen Transplantation Service ApS, Copenhagen, DK or at University of Lübeck, Germany. The chondrocyte cells were trypsinized using trypsin EDTA for 5 to 10 minutes and counted using Trypan Blue viability staining in a Bürker-Türk chamber. The chondrocyte cell count was adjusted to 5×10⁵ chondrocyte cells per milliliter. One NUNCLON™ plate was uncovered in the Class 100 laboratory.

The Chondro-Gide® support matrix, as in Example 1, was cut to a suitable size fitting into the bottom of a well in the NUNCLON™ cell culture tray. In this case a circle of approximately 4 cm in diameter was placed under aseptic conditions on the bottom of the well.

After three weeks, the chondrocyte cells were transferred from the growth media to the transplant media described above, and approximately 5×10⁶ cells in 5 ml transplant media were placed directly on top of the support matrix and dispersed over the surface of the support matrix. The plate was incubated in a CO₂ incubator at 37° C. for 3 weeks.

The support matrix holding the grown chondrocyte cells was then incubated with collagenase for 16 hours. The support matrix holding the chondrocyte cells was then centrifuged. Cells were seeded on a NUNCLON™ plate and an aliquot counted using Trypan Blue viability staining in a Bürker-Türk chamber. A microphotograph thereof is shown in FIG. 11C. The total calculated cell number was found to be 6×10⁶ and the viability was >95%.

### EXAMPLE 11

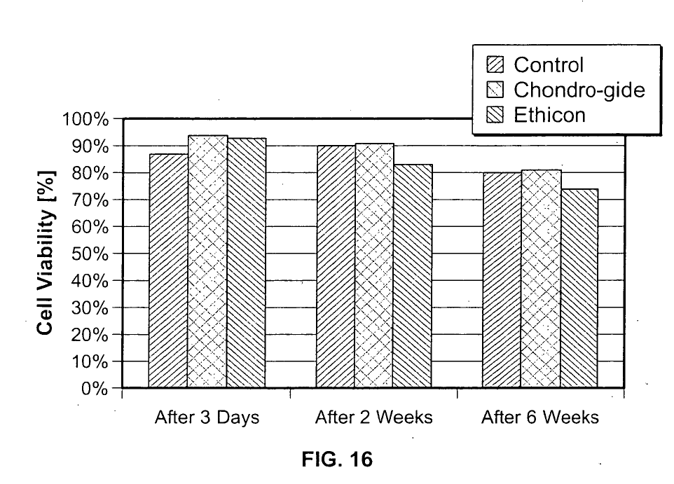

The present example describes a test of the toxicity and biocompatability of a membrane prepared according to U.S. Pat. No. 4,902,508; (assigned to DePuy, a subsidiary of Ethicon, Inc.; the DePuy membrane or “Ethicon” in FIG. 16) and Chondro-Gide® membrane (Ed Geistlich Sohne, Geistlich Pharma AG, Wolhusen, Switzerland). The viability and number of chondrocyte cells adhered to the DePuy and Chondro-Gide® membranes for three days, two weeks, and six weeks, was determined by visually counting the number of cells adhered to the membranes.

The DePuy membrane was tested with the Chondro-Gide® membrane as positive control, and a negative control using the same method but without any membrane.

Previously frozen human chondrocyte cells (14 million cells) were thawed and washed, and the cell number and viability were determined. 3.2 million cells were recovered after thaw at 87% viability. 1.6 million cells were added to each of two tissue culture flasks at a concentration of 5.3×10⁴ cells per milliliter, and incubated at 37° C. for three days. The resulting cell number and viability for the flasks were 3.5 million cells, with 98% viability and 3.6 million cells, with 93% viability, respectively.

Cell count and viability were analyzed for six samples of each membrane, and six samples of the control group without any membrane. The membranes were analyzed at three days, two weeks, and six weeks. The samples of the DePuy and Chondro-Gide® membranes were cut into one inch squares. Because of the dry consistency of the DuPuy membrane, cutting the membrane can be somewhat difficult.

Chondrocytes were trypsinized and cell viability and cell number were determined as indicated above. The cells were pelleted by centrifugation and resuspended to a concentration of 1 million cells per milliliter.

The DePuy and Chondro-Gide® membranes were washed twice with phosphate buffered saline (PBS), having a pH of 7.17. Each membrane was inserted into a well of a culture dish. One hundred microliters of a chondrocyte cell suspension at a concentration of 1 million cells per milliliter was applied to each piece of membrane and in the bottom of six wells with no membrane. Additional culture medium (3 milliliters) was added to each well. The culture plates were incubated for at least three days at 37° C.

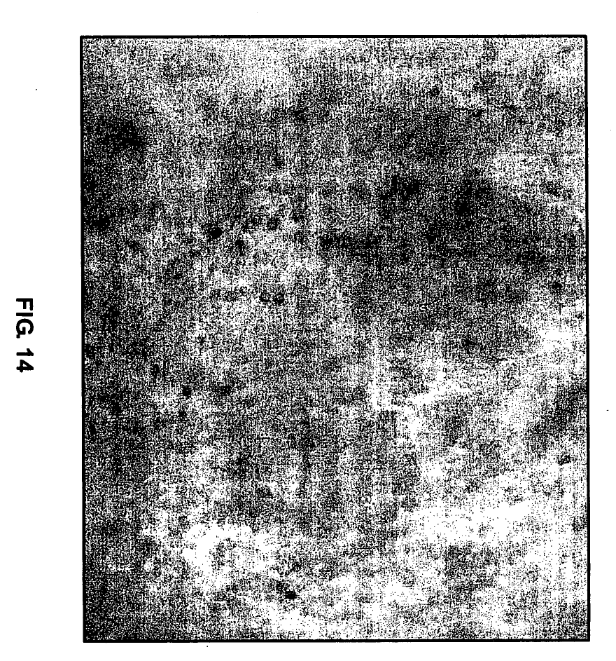

FIG. 14 illustrates chondrocyte cells adhered to the DePuy membrane. Cells were harvested and counted at three days, two weeks, and six weeks.

The DePuy and Chondro-Gide® membranes were treated with an enzyme solution containing a mixture of 2 milliliters of 0.25% trypsin and 1 milliliter of collagenase (a total of 5000 units) to dissolve the membranes so cell count and viability could be determined. Once dissolved, the chondrocyte cells were harvested by centrifugation and counted. The length of treatment with the enzyme solution varies with the type of membrane. For the DePuy membrane, collagenase digestion was much longer than for the Chondro-Gide®. The DePuy membrane was not completely dissolved after 2 hours of digestion while the Chondro-Gide® membrane completely dissolved in about 1 to 1.5 hours. To avoid cell stress, collagenase digestion should not proceed for more than 2 hours.

The control group of chondrocytes were trypsinized, washed, and pelleted by centrifugation. The cells were then resuspended in DMEM medium and the final cell number was ascertained.

The results of the experiment are as follows. There was not substantial variation in the viability of control cells compared with cells grown on the DePuy and Chondro-Gide® support matrices. As shown in FIG. 16, at three days, cell viability in the control, Chondro-Gide® support matrix, and the DePuy support matrix was high, at about 87% for the control, about 94% for the Chondro-Gide® support matrix, and 93% for the DePuy support matrix. At the two-week interval, viability for the control group was about 90%, about 91% for the Chondro-Gide® group, and about 83% for the DePuy group. At six weeks, about 80% of the cells were viable using control conditions, about 81% of the cells were viable on the Chondro-Gide® membrane, while about 74% of cells were viable using DePuy support matrix conditions. Thus, there was not a wide-spanning variation in the viability of the cells in the different growth conditions.

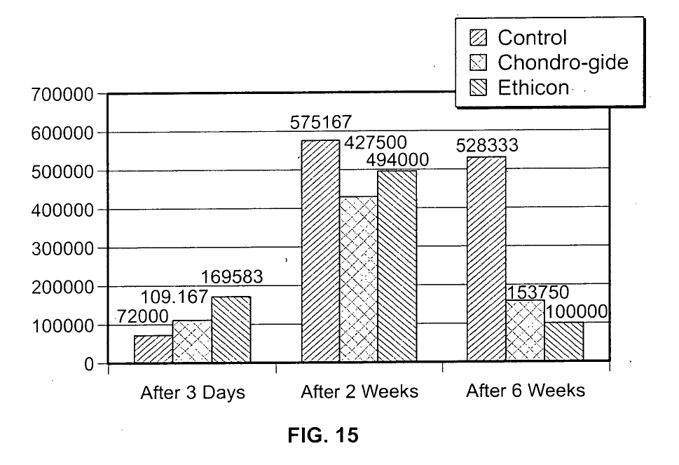

Actual cell number was not significantly affected by the DePuy and Chondro-Gide® support matrices until late in culture. As shown in FIG. 15, at the three-day time point, control cells amounted to about 72,000 cells while Chondro-Gide® cells were about 109,167 and the DePuy cells amounted to about 169,583 cells on the support matrices. At two weeks, control cells proliferated about eight times to 575,167, while Chondro-Gide® cells nearly quadrupled in number to 427,500 and the DePuy cells nearly tripled in number to 494,167. Finally, at six weeks, the number of control cells decreased slightly to about 528,333 while the number of Chondro-Gide® cells and the DePuy cells decreased dramatically to 153,333 and 100,833, respectively.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

We claim:

1. An article comprising a membrane having at least one layer having a porous surface and including submucosal intestine tissue, and cells adhered to said layer.

2. An article according to claim 1, wherein said cells are chondrocyte cells.

3. An article according to claim 1, wherein said membrane is cell-free.

4. An article according to claim 1, wherein said membrane is collagen.

5. An article according to claim 1, wherein said membrane is Type I and Type III collagen.

6. An article according to claim 1, wherein said membrane is resorbable.

7. An article according to claim 1, wherein said chondrocyte cells are autologous.

8. An article according to claim 1, further comprising biocompatible adhesive adjacent said membrane.

9. An article according to claim 1, wherein said membrane is adapted to be disposed over the articular cartilage defect.

10. An article according to claim 1, wherein said membrane is adapted to be disposed in the articular cartilage defect.

11. An article according to claim 1, wherein said membrane is disposed over the articular cartilage defect.

12. An article according to claim 1, wherein said membrane is disposed in the articular cartilage defect.
